(19) United States
(12) Patent Application Publication
(10) Pub. No.: US 2025/0195257 A1
(43) Pub. Date: Jun. 19, 2025

(54) COLLAR DRAPE AND BARRIER FOR AN OSTOMY APPLIANCE

(71) Applicant: Ultra Wound Care, LLC, BRENTWOOD, TN (US)

(72) Inventors: Thomas R. GALBIERZ, Brentwood, TN (US); Michael A. GALBIERZ, St. Louis, MO (US)

(21) Appl. No.: 18/980,969

(22) Filed: Dec. 13, 2024

Related U.S. Application Data
(60) Provisional application No. 63/611,304, filed on Dec. 18, 2023.
(60) Provisional application No. 63/554,115, filed on Feb. 15, 2024.

(51) Int. Cl. A61F 5/443; A61F 5/44; A61F 5/448; A61F 5/449

(52) U.S. Cl. CPC A61F 5/443; A61F 5/4408; A61F 5/448; A61F 5/449

(57) ABSTRACT

A collar drape and a barrier are disclosed which can be used with an ostomy appliance to form an enhanced seal with a wearer's stoma. The collar drape comprises a collar drape body defining a collar drape opening sized to be equal to or slightly smaller than an outer diameter of the neck of the ostomy appliance. The barrier comprises a barrier base defining a barrier opening spaced from a perimeter of said barrier. The barrier base is formed from a material to which said ostomy appliance flange can be removable adhered. The barrier base defines a perimeter greater than a perimeter of a flange of the ostomy appliance.

### CROSS-REFERENCE TO RELATED APPLICATIONS

This Application claims priority to U.S. App. No. 63/611,304 filed Dec. 18, 2023, and entitled “Collar Drape For An Ostomy Appliance”, and US App. No. 63/554, 115 filed Feb. 15, 2024 and entitled Collar Drape And Barrier For An Ostomy Appliance, both of said applications being incorporated herein by reference.

### STATEMENT REGARDING FEDERALLY SPONSORED RESEARCH OR DEVELOPMENT

Not Applicable.

### BACKGROUND

This application relates generally to adhesive devices that help maintain and/or improve the seal of an ostomy appliance to a wearer's skin to reduce, contain, or substantially eliminate leakage of bodily fluid(s) to thereby provide better care for skin of the peristomal plane surrounding the wearer's stoma.

As is known, there are two types of ostomies-loop ostomies, when a loop of, for example, colon or intestine, is pulled through an opening in the abdomen and opened up to form a loop stoma, and an end ostomy, when, for example, the end of the colon or small intestine extends through an opening in the abdomen to form an end stoma. Ostomy appliances surround and enclose the stoma to collect output from the stoma. Ostomy appliances comprise an ostomy bag/pouch which is secured to the top side or surface of a base or flange, which, in turn, is secured to the abdomen around the ostomy by means of an adhesive applied to the backing of the base. The base has a hole sized to surround the wearer's stoma and which is aligned with an opening into the bag/pouch to thereby place the stoma in communication with the interior of the bag/pouch. Typically, the base will be formed from a material which can be cut, so that the hole can be formed to the appropriate size for the stoma to reduce the amount of peristomal skin exposed between the stoma and the perimeter of the hole. Waste, such as fecal matter, thus passes from the stoma into the bag/pouch through an opening in the bag/pouch. This stoma-receiving opening is near the top of the bag/pouch on a back surface of the bag/pouch. The bag/pouch usually also includes a second opening at its bottom which allows for the bag/pouch to be emptied without the need to remove the bag/pouch from the base.

Sometimes, the seal of the base around the stoma is not sufficient, and ostomy output (which can be acidic and/or corrosive, especially for an ileostomy) can contact the skin around the stoma, causing the peristomal skin to become irritated or inflamed. Sealing problems can occur, for example, when the stoma is sunken relative to the abdominal surface or when the skin surrounding the stoma (the peristomal plane) is not flat, such as when there are folds or creases in the skin. Such physiology can create an area around the stoma that is sunken relative to the rest of the abdomen, making it difficult for the base of the appliance to seal properly against the wearer's skin and can leave gaps or cavities into which fluids from the stoma can leak. Various manufacturers have attempted to solve this issue by providing different configurations of bases and different methods for mounting the bag/pouch to the base. However, it would be desirable to provide a product that can enhance the seal of the base, and which is independent of the ostomy appliance.

### SUMMARY

Briefly, two adhesive devices, a collar drape and a barrier, are provided for use with an ostomy appliance to improve the seal surrounding a stoma to reduce the potential of leakage of output from the stoma. The ostomy appliance in general comprises a base or flange adherable to skin and a bag/pouch operatively connected to the appliance base at a neck.

The collar drape and barrier can be provided as a kit with an ostomy appliance, enabling a wearer to use the barrier and collar drape in three ways. In a first mode, only the collar drape is used with the ostomy appliance. In a second mode, only the barrier is used with the ostomy appliance. And in a third mode, the barrier and collar drape are used together (or in combination) with the ostomy appliance. In the first mode, the collar drape is applied directly to the patient, and the ostomy appliance is applied (adhered) to the collar drape. In the second mode, the barrier is applied directly to the patient and the ostomy device is applied (adhered) to the barrier. Lastly, in the third mode, the barrier is applied to the patient, the ostomy appliance is applied to the patient over the barrier, and the collar drape is applied over the flange of the ostomy appliance. In this last mode, the barrier can also cover a portion (or all of) the collar drape. The manner of use will be determined by the wearer's needs.

Turning initially to the collar drape, the collar drape comprises a body formed from an adhesive assembly. The adhesive assembly comprises an elastic stretchable flexible material which in turn comprises a top layer, a reinforcing layer, and an adherent layer having an adherent adapted to secure the drape to skin of the wearer.

The collar drape body defines an opening sized to be equal to or slightly smaller than an outer diameter of the neck of the ostomy appliance. The opening is generally centered vertically along a horizontal axis (HA) or is vertically spaced above the horizontal axis (HA) of the body. In this latter instance, a distance (D1) from a center of the opening to a bottom edge of the collar drape is greater than a distance (D2) from the center of the opening to a top edge of the collar drape.

In accordance with an aspect of the collar drape, the opening is sized to permit the ostomy bag/pouch to pass through the collar drape opening during application and use of the collar drape.

In accordance with an aspect of the collar drape, the collar drape comprises a release liner removably adhered to the adherent layer. The release liner defines a removable border in the release layer surrounding the opening of the collar drape; whereby removal of the border enables the opening of the collar drape to expand when the drape is positioned about an ostomy appliance while the release liner remains in place over the remainder of the collar drape.

In accordance with an aspect of the collar drape, the collar drape of includes arms which extend from opposite sides of the drape body. The arms can be of the same length. Alternatively, one arm can have a length that is shorter than the other arm. In this latter instance, the shorter arm is preferably wider than the longer arm.

In accordance with an aspect of the collar drape, the collar drape has a length from a top edge to a bottom edge that is equal to at least 70% of the length of the ostomy bag/pouch.

In accordance with an aspect of the collar drape, the lower distance (D1) from the center of the opening to the bottom edge of the drape is equal to at least 70% of the length of the ostomy bag/pouch from a stoma receiving aperture of the ostomy bag/pouch to a bottom of the ostomy bag/pouch.

In accordance with an aspect of the collar drape, the drape has a plush upper surface. Preferably, the plush surface is adapted to frictionally engage a plush surface of the ostomy bag/pouch to reduce movement of the ostomy bag/pouch relative to the wearer.

In accordance with an aspect of the collar drape, the plush upper surface is rough when rubbed in a direction generally parallel to a horizontal axis of the collar drape but smooth when rubbed in a direction generally parallel to a vertical axis of the collar drape.

In accordance with an aspect of the collar drape, the collar drape can be provided with a strap which is removably securable to the collar drape. This strap comprises an elastic central section and opposed ends which are adapted to removably engage the plush surface of the collar drape. The strap can be applied to the collar drape (1) to extend between the ostomy bag/pouch and the collar drape to vertically support to the ostomy bag/pouch during use or (2) to extend over the ostomy bag/pouch to restrain movement of the ostomy bag/pouch relative to the collar drape.
In accordance with an aspect, the strap is elastic and has a length at least when stretched that is greater than a side-to-side width of a base of the ostomy appliance, wherein when the strap is applied to the collar drape below the neck of the ostomy appliance, the strap will add pressure to the base of the ostomy appliance below the neck.

In accordance with an aspect of the collar drape, due to its elasticity, when the collar drape is applied to a wearer in a stretched state, the collar drape will generate compressive forces which are applied/directed to the flange and neck of the ostomy appliance to enhance a seal of the ostomy appliance about the stoma.

Turning to the barrier, the barrier comprises a barrier base formed from a substrate and a barrier adhesive covering a bottom surface of the substrate.
The barrier base has an upper surface, a lower surface, and an opening spaced from a perimeter of the barrier. The barrier base is formed from a material, preferably a film comprising polyurethane, polyethylene, or combinations thereof, to which the ostomy appliance flange can be removable adhered. The barrier base defines a perimeter greater than a perimeter of the ostomy appliance flange, such that when the ostomy appliance flange is adhered to the barrier base, the barrier base will extend beyond the perimeter of the ostomy appliance flange.
The barrier adhesive is stronger than the adhesive of the ostomy appliance flange, whereby the ostomy appliance can be removed from the barrier without removing the barrier from the wearer.

In accordance with an aspect of the barrier, the opening of the barrier is defined by a rolled or folded over inner portion of the barrier defining an inner surface which is sized to engage the stoma about the periphery of the stoma when applied, such that the adhesive of the barrier seals with the stoma.

In accordance with an aspect of the barrier, an adhesive tape secures the inner portion of the barrier in the rolled or folded over position.

In accordance with an aspect of the barrier, the barrier further comprises an annular ring, which is preferably separate from the barrier base. The ring has an inner surface defining an opening sized and shaped to engage and seal against a stoma, and a bottom surface having an outer diameter greater than a diameter of the barrier opening and an inner diameter no greater than the diameter of the barrier opening. The ring is adapted to be adhered to the upper surface of the barrier base. Preferably, the annular ring preferably being made form a hydrocolloid adhesive.

In accordance with an aspect, the ring is generally triangular in cross-section, wherein the inner surface of the ring defines a generally vertical inner wall. The ring includes a sloping upper surface extending from a top of the wall to an outer edge of the bottom surface.

In accordance with an aspect of the barrier, the barrier adhesive is a silicone gel or acrylate gel.
In accordance with an aspect, the barrier adhesive has an adhesion of about 1.6 N per 25 mm.
In accordance with an aspect, the barrier adhesive is applied to the bottom surface of the substrate at a weight of approximately 250 grams per square meter.

In accordance with an aspect of the barrier, the barrier further comprises a lower release liner sized to cover and protect the barrier adhesive, wherein the lower release liner preferably formed from siliconized paper.

In accordance with an aspect of the barrier, the barrier further comprises an upper release liner covering the upper surface of the substrate. This upper release liner is preferably formed from a low-density polyethylene (LDPE).

In accordance with an aspect of the barrier, the barrier comprises a first portion and a second portion; the first and second portions each having an inner edge defining an arcuate cutout; the cutouts of the first and second portions, in combination, defining the opening of the barrier.

The various aspects of the collar drape can be combined in any desired combination. Similarly, the various aspects of the barrier can be combined in any combination.

When provided as a kit, the kit includes an ostomy appliance, the barrier as described above, and the collar drape as described above.

In accordance with an aspect of the kit, in the mode described above when the barrier is used in conjunction with the collar drape, the opening defined by the collar drape is larger than the opening defined by the barrier but smaller than an outer diameter of the barrier.

### BRIEF DESCRIPTION OF THE DRAWINGS

FIGS. 1A-B comprises schematic vertical cross-sectional views of a one-piece and a two-piece ostomy appliance, respectively;

FIG. 2 is front plan view of a collar drape for an ostomy appliance;

FIG. 3 is a rear or back plane view of the collar drape;

FIG. 4 is a plan view of an alternative collar drape in which a hole of the collar drape is generally centered in the body of the collar drape and two arms of the collar drape are of different dimensions and shape;

FIG. 5 is an enlarged schematic cross-sectional view of the material from which the collar drape is made;

FIG. 6 is a plan view of the collar drape with an ostomy appliance shown illustratively;

FIG. 7 is an illustrative plan view similar to that of FIG. 6, but with the addition of an elastic strap which will increase pressure on the base of the ostomy appliance below a neck of the appliance;

FIG. 8 is a schematic cross-sectional view of the collar drape applied to a wearer in conjunction with a schematically shown ostomy bag/pouch;

FIG. 9 is a cross sectional view of a one-piece ostomy appliance secured to a wear's skin about a stoma in conjunction with the collar drape;

FIG. 10 is a view similar to FIG. 9, but with the ostomy appliance being of the type provided with support tabs;

FIG. 11 is a cross-sectional view of a two-piece ostomy appliance secured to a wear's skin about a stoma in conjunction with the collar drape, wherein the ostomy appliance is of type provided with support tabs;

FIG. 12 is a schematic cross-sectional view of a collar drape applied to a stoma with an ostomy appliance and an optional barrier;

FIG. 13 is an enlarged view taken along the circle 2 in FIG. 12;

FIG. 14 is a view similar to FIG. 12, but with an alternate barrier;

FIG. 15 is an enlarged view of the alternative barrier taken at the dotted oval in FIG. 14;

FIGS. 16A-B are plan views of a further planar barrier;

FIG. 17 is a plan view of a 2-piece planar barrier;

FIG. 18A is a plan view similar to the barrier of FIG. 16, but with a slightly different shape;

FIG. 18B is a plan view of the barrier showing the relative position of the two parts relative to each other when in use;

FIG. 19 is a plan view of an assembly (in use) comprised of the collar of FIG. 1 and the barrier of FIGS. 17A-B in conjunction with an ostomy appliance; and

FIG. 20 is a schematic plan view of a barrier in use with an ostomy appliance, but without a collar.

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what we presently believe is the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

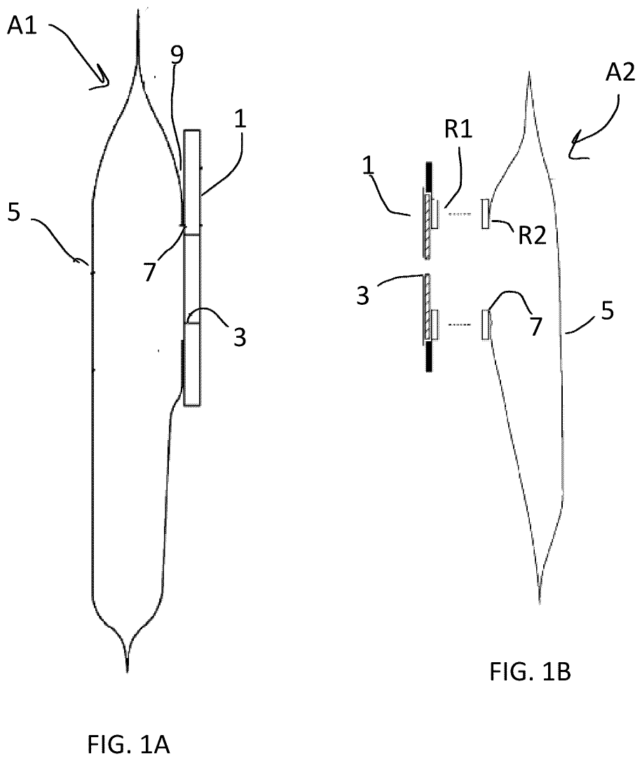

FIGS. 1A and 1B are schematic vertical cross-sectional views of generic one-piece and two-piece ostomy appliances, respectively. Both the one-piece and two-piece ostomy appliances A1, A2 comprise a flange or base portion 1 which adheres to the wearer's skin and defines an opening 3 which is selectively sized to fit about the wearer's stoma. A bag/pouch 5 is then secured to the base or flange 1. The bag/pouch 5 includes an opening 7 in alignment with the opening 3 in the appliance flange such that the stoma will extend into the interior of the bag/pouch, such that output from the stoma (colon, ileus, or urethra) will be deposited in the bag/pouch. Typically, the base 1 has a hydrocolloid layer on its back surface which adheres the flange to the skin. This hydrocolloid layer provides a relatively weak adherence to the skin, to reduce damage to the skin when the appliance is removed. In the one-piece appliance A1, the bag/pouch 5 is permanently fixed to the flange 1 and there is a narrow connection or neck area 9 at the junction of the bag/pouch 5 and flange 1. In the two-piece appliance A2, the flange 1 and bag/pouch 5 each have a ring R1, R2 surrounding their respective openings. The two rings are sized and shaped to be matingly connected, to thus (removably) connect the bag/pouch 5 to the flange 1. In this instance, the rings R1, R2, in combination, can define a short neck. In some appliances, tabs can extend from the ring of the bag/pouch which allow for connection of a belt to further secure the bag/pouch to the wearer. The outer circumference of the necks/connectors of the one- and two-piece ostomy appliances is typically in the range of about 2-4 inches. The openings 3,7 through which the stoma extends can have a diameter of between 10 mm (˜0.4″) and 60 mm (˜2.4″) and are sized according to the size of the stoma. Depending on how the flange 1 is made, the size of the opening 3 can be fixed or the flange material can allow for sizing of the opening (for example, by cutting the material to increase the size of the opining 3).

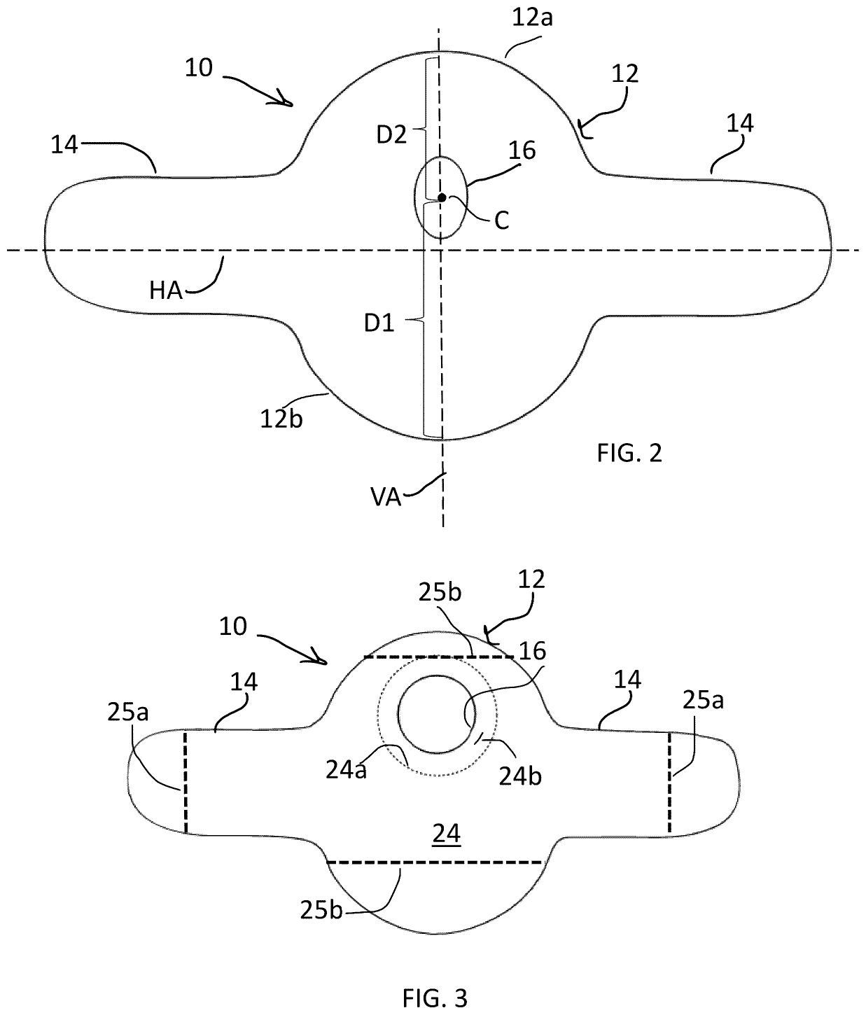

Turning to FIG. 2, a collar drape 10 for the ostomy appliance A1, A2 is shown. As discussed below, the collar drape 10 is provided to be used with an ostomy appliance A1, A2 to enable forming of a better seal around the stoma and to thus reduce the potential for irritation of the peristomal skin. The collar drape 10 comprises a body 12 that, as shown, is generally oval in shape and is elongated vertically. The body includes an upper edge 12a and a lower edge 12b, both of which are shown to be generally arcuate (but could be any desired shape). Inner and outer arms 14 extend from each side of the body 12, with the upper and lower edges of the arms intersecting the lower and upper ends of the upper and lower edges 12a,b, respectively. As will become apparent below, in use, the inner arm extends toward the center of the wearer's body and the outer arm extends toward the wearer's side. The arms 14 are preferably generally centered vertically relative to the body 12. As depicted, collar drape 10 defines a vertical axis VA and a horizontal axis HA. The collar drape 10, as shown, is generally symmetrical about the vertical axis VA with the arms 14 being of about the same length. However, the upper and lower edges 12a ,b of the body 12 may not be identical, and thus, the collar drape 10 may not be symmetrical about the horizontal axis HA. Although the collar drape 10 could be symmetrical about the horizontal axis HA, preferably the portion below the horizontal axis HA is longer than the portion above the horizontal axis. Further, although the arms 14 are shown to be of the same length, the outer arm could be shorter and wider than the inner arm, in which case the collar drape would not be symmetrical about the vertical axis VA (although the body 12 could be). Further, if desired, the arms could be off center relative to the body and the horizontal axis HA (either in the same or opposite directions).

A collar drape opening 16 is formed in the body 12. The collar drape opening 16 is sized to fit about the base of the neck 9 or connection of the ostomy bag/pouch to mimic the neck base profile. The collar drape opening 16 is shown to be generally oval in shape, with a vertical length of about 2″ to about 3″ and a horizontal width of about 1¾″ to about 2½″, such that the collar drape opening is generally vertically oriented. Although shown to be generally oval, the collar drape opening 16 can be any other desired shape. At least the width of the collar drape opening 16 (in a direction parallel to the horizontal axis HA) is slightly less than the outer circumference of the neck 9. The length of the collar drape opening 16 (in a direction parallel to the vertical axis VA) is also preferably less than the outer circumference of the neck, but can be greater. Preferably, the width of the collar drape opening 16 is about 10% to about 20% smaller than the neck outer circumference and the length of the opening is about 0% to about 10% smaller than the neck outer circumference. Further, as seen, the collar drape opening 16 is generally centered horizontally relative to the body 12 (i.e., it is generally bisected by the vertical axis VA). However, the collar drape opening 16 is offset from the horizontal axis VA. As shown, a bottom of the collar drape opening 16 is at, or above, the horizontal axis HA. Thus, a distance D1 from the center C of the collar drape opening 16 to the bottom edge of the collar drape is greater than a distance D2 from the center C of the collar drape opening 16 to the top edge of the collar drape. Preferably, the distance D1 from the center of the collar drape opening 16 to the bottom of the collar drape is preferably at least about 70% (or about 80%) of the distance as from the center of the appliance opening to the bottom of the bag/pouch. For example, the distance from the center of the collar drape opening 16 to the bottom edge of the collar drape could be about 3″ to about 6″. The distance D2 from the center C of the opening to the top edge 12a of the collar drape is preferably about equal to the height of the bag/pouch B above the bag/pouch's stoma-receiving opening. For example, the distance D2 can be about 3″ to about 4″. As can be appreciated, the total height (or vertical length) of the collar drape 12 is preferably about equal to the length of the ostomy bag/pouch. Thus, as shown schematically in FIGS. 6 and 7, most, if not all, of the bag/pouch will be separated from the wearer by the collar drape body 12. Preferably, the height of the collar drape is at least 70% of the length of the bag/pouch.

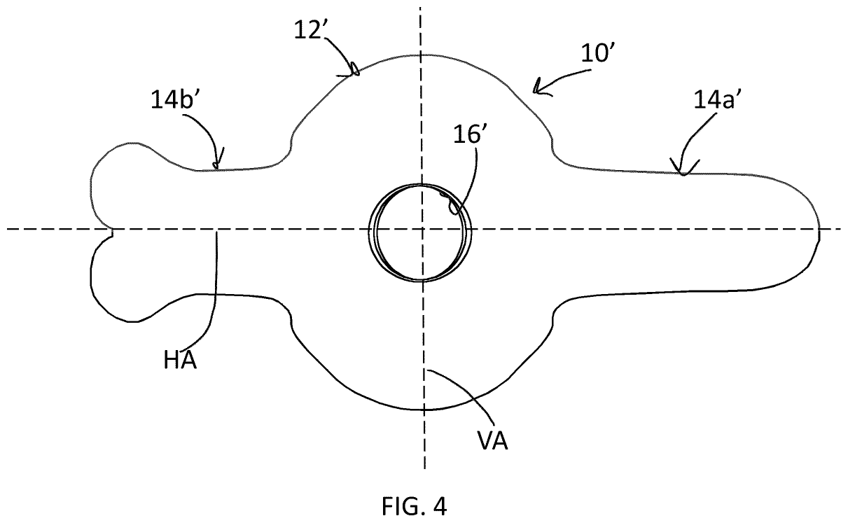

An alternative collar drape 10′ is shown in FIG. 4. Like the collar drape 10 of FIGS. 2-3, the collar drape 10′ includes a body 12′ with a collar drape opening 16′ and arms 14a′ and 14b′ extending from opposite sides of the body. However, unlike the collar drape 10, the collar drape 10′ is symmetrical about its horizontal axis HA with the collar drape opening 16′ having a center that is generally aligned with the intersection of the horizontal and vertical axis HA and VA. Further, as seen, the arms 14a′ and 14b′ are different. The arm 14a′ is elongate with upper and lower edges that are generally parallel or slightly canted towards each other. The arm 14b′, on the other hand, is shorter and wider than the arm 14a′ and has upper and lower bulges at its ends, giving it a generally bone shaped appearance.

### The Preferred Material From Which the Re-appliable Collar Drape is Made

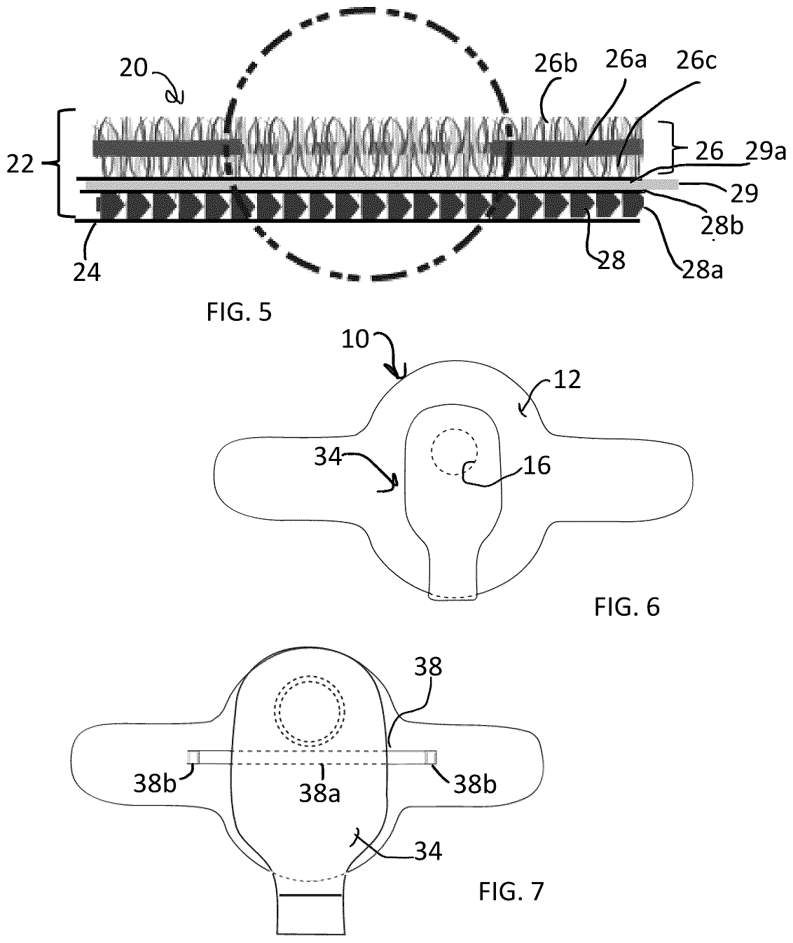

FIG. 5 schematically shows a cross-section of the preferred material 20 from which the collar drape 10 is formed. The material 20 is an adhesive assembly comprising an elastic, stretchable, and flexible material 22 and a release liner 24 removably secured to the flexible material 22. The flexible material 22 comprises a top layer 26, a reinforcing layer 29, and an adherent layer 28. The release liner (or bottom ply) 24 is removably attached to the adherent layer 28 to protect the adherent layer until the flexible material is put into use. When the release liner is removed, the adherent layer will, as described below, removably adhere the collar drape 10 formed from the material 22 to a surface, such as a patient's skin.

The top layer 26 preferably comprises a stretchable, elastic base material 26a which, in a preferred embodiment, is formed from a knit material, a woven material, or a tricot material. Preferably the top layer 26 is elastic and stretchable in all directions, rather than in just one direction. The material which forms the base material 26a can comprise man-made fibers, natural fibers, or a combination thereof. Preferably, the base material 26a is formed from a combination of fibers, at least a portion of which are elastic fibers, such as spandex (a polyether-polyurea copolymer), such as, for example, Lycra®. If formed from a combination of fibers, the elastic fibers can be combined, intertwined, or formed with, for example, natural fibers, such as cotton fibers, or man-made fibers, such as nylon or spandex, to form the thread or yarn from which the material for the top layer 26 is formed. The elastic fibers provide the stretchability and elasticity of the top layer 26. The base material can, for example, be about 5% to about 95% nylon and about 95% to about 5% spandex. In a preferred embodiment, the base material is about 90% nylon and about 10% spandex. Illustratively, the nylon can be SD Nylon and the spandex can be 40 Denier Spandex. Further, the material can comprise different types of nylon, such as “50/17 SD” nylon and “20/1 SD” nylon. The two types of nylon can be combined in ratios of about 4:1 “50/17 SD” nylon: “20/1 SD” Nylon to about 1:4 “50/17 SD” nylon: “20/1 SD” nylon, and preferably about 3.2:1 “50/17 SD” nylon: “20/1 SD” nylon. In a preferred embodiment, the material comprises about 69% “50/17 SD” nylon, about 21% “20/1 SD” nylon, and about 10% 40 denier spandex. Further, in this embodiment, the base material 26a is composed of materials which are generally inert and generally biocompatible. Fibers other than nylon and spandex can be used. For example, the stretchable (spandex) component of the flexible material could comprise silicone and/or natural rubber; and the nylon could be replaced with polyethylene and/or polyurethane. The base material can be made of other types of natural fibers or man-made/industry-made fibers (or combinations thereof). For example, the other man-made/industry-made fibers can include carbon fibers, expanded polytetrafluoroethylene (commonly known by the brand name, Gore-Tex®), or other synthetic and/or conductive fibers. The top layer 26 can be made, as noted above, by weaving or knitting the fabric top layer. If the top layer is made from man-made materials, the top layer can be formed by 3D printing.

The top layer 26 further comprises upper fibers, pile, or napping 26b and lower fibers, pile, or napping 26c which extend from the upper and lower surfaces, respectively, of the base material 26a to define upper and lower napped or plush surfaces of the top layer 26. The fibers/pile/napping 26b, c can be formed by brushing or abrading the surfaces of the base material 26a. As such, the upper and lower napped surfaces 26b, c have the same composition as the base material 26a. The upper and lower fibers/pile/napping 26b, c, depending on how they are formed, can be straight, curved, twisted, etc. Further, the fibers/pile/napping may not be uniformly shaped. The lower fibers 26c extending from the bottom of the base material 26a greatly increases the surface area of the bottom of the top layer 26 as compared to a “smooth” (i.e., un-napped) material.

The upper and lower pile or napping 26b, c can be formed by brushing the base material 26b in a single direction (preferably in a machine direction of the material). We have found that when the pile/napping is formed in this manner, when the material 22 is folded over on itself along a line generally parallel to the direction of brushing, such that upper pile contacts upper pile, the two layers of material can move relative to each other in a direction generally parallel to the fold relatively freely, and movement in this direction (generally perpendicular to the direction of brushing) is like the fabric is moving along track. On the other hand, when the fabric layers are moved relative to each other in the machine direction (i.e., parallel to the direction of brushing), when the fabric is moved in the same direction as the brushing was performed, the fabric moves freely. However, when moved against each other in a direction opposite to the direction of brushing, there is some friction, like the material is grabbing at itself.

The reinforcing layer 29 is positioned below the top layer 26, and is adhered to the top layer by an adhesive, such as an acrylate adhesive or binder 29a, applied to the top side of the reinforcing layer. This reinforcing layer 29 can be made from a polyurethane film or a polyethylene film and can add some slight rigidity to the material 22, which will make the material less “floppy” and a bit easier to handle, especially when wearing gloves. Although the reinforcing agent will add some rigidity to the material 22, the reinforcing layer is stretchable, and preferably, elastic, in all directions, such that use of the reinforcing layer 29 will not diminish the stretchability or elasticity of the top layer 26. If a binder is used, the binder can be, for example, an acrylic or acrylate adhesive which will bond to the reinforcing layer and which will secure the reinforcing layer to the cover layer 26. The adhesive binder 29a which adheres the reinforcing layer 29 to the top layer 26 extends into the pile 26c (or the pile 26c extends into the binder) to increase the contact surface area of the upper layer with the binder. The binder also bonds with the reinforcing layer, such that the reinforcing layer cannot easily be removed from the top layer 26.

The adherent layer 28, as noted, is secured to the bottom surface of the reinforcing layer 29. As disclosed in US20230046036, which is incorporated herein by reference, the adherent layer 28 comprises at least an adherent 28a which adheres to, but does not aggressively bond to, skin and is gentle on the skin during long term wear as well as during removal. Thus, the collar drape 10 is removably attached to a patient's skin, and damage to the skin during removal of the collar drape 10 is minimized by the use of the adherent. The adherent is preferably a silicone gel material or a hydrogel adhesive, and has viscoelastic and/or high co-adhesive properties. It can be applied to the bottom surface of the reinforcing layer 29 at a heavy (high) coat weight thickness of about 100-500 g/m², and preferably about 250-300 g/m². Such adhesives are typically composed of a very lightly cross-linked silicone elastomer whose polymer network has been swollen with silicone fluids. The silicone gel has elastic characteristics, is stretchable in any direction, and will elongate with the top layer 26 and reinforcing layer 29. That is, as the silicone gel elongates, the silicone gel layer becomes thinner. The thinning of the silicone gel layer is constant, and thus, the silicone gel layer does not get thinner in just one area, but rather thins uniformly. Thus, the silicone gel layer is colinear. The same is true for the top layer 26 and the liner 29.

Preferably, the adherent layer includes a binder 28b that secures the silicone gel adhesive to the bottom surface of the reinforcing layer 29. The binder 28b is preferably the same as the binder 29a, i.e., an acrylate adhesive. The binder 28b bonds to both the silicone gel 28a and the reinforcing layer 29 to securely fix the adhesive/silicone gel layer 28a to the reinforcing layer 29. If the binder 28b is used, the binder is covered by the silicone gel 28a, such that the acrylate adhesive binder 28b will not come into contact with the patient's skin during use. The reinforcing layer 29 is preferably coextensive with at least the colinear adhesive layer 28, and more preferably coextensive with the top layer 26.

The release liner 24, as noted, is removably secured to the adherent layer 28. The release liner 24 is preferably made from a generally non-stretchable material, which is held to the adherent 28a by the natural adhesive qualities of the silicone adherent. The adhesive force of the silicone adherent 28a is substantially less than the adhesive force of the binder 28b, and thus, removal of the release liner 24 from the silicone adherent will not pull the silicone adherent from the top layer 26 or the reinforcing layer 29. The material for the release liner 24 is chosen by design to be a material which has low adhesion force with respect to the silicone adherent. The release liner 24 does not directly contact the top layer 26 (or reinforcing layer 29). Thus, the release liner 24 is removably adhered to the bottom of the material 22 by the silicone adherent layer 28. The release liner 24 could have a coating, for example, of fluorosilicone applied to the side of the release liner which is in contact with the silicone gel adherent. This will facilitate removal of the release liner 24 from the material 22. Additionally, the release liner could extend beyond the perimeter of the material construction 22 for ease of removal and then storing of a device when not in use. In an alternative, the device 10, 10′ could be provided with tabs or perforations, as disclosed, for example, in U.S. Pat. No. 9,427,222, which is incorporated herein by reference, to facilitate removal of the release liner 24 from the material 22.

Returning to FIG. 3, the collar drape 10 preferably includes a perforated circle 24a in the release layer 24 which surrounds (and can be concentric with) the collar drape opening 16. The collar drape can be provided with a tab to facilitate removal of the backing defined by the perorated circle 24a.

The collar drape 10 can also be provided with slits 25a positioned toward the ends of the arms and slits 25b which extend horizontally across the body 12 above and below the collar drape opening 16. The upper and lower slits 25b are shown to extend across the body 12 above and below the lower edges of the arms 14. The slits 25a are generally parallel to the vertical axis VA and the slits 25b are generally parallel to the horizontal axis HA. The slits 25a,b extend only through the release layer 24. When, for example, the ends of the arms are grasped and pulled to stretch the material 22, the release layer separates along the slits allowing for easier removal of the backing layer from the body. The slits 25a,b thus essentially define grasping areas on the body 12 of the collar drape 10. Although the slits 25a,b are shown to extend vertically through the arms and horizontally through the body 12 of the collar drape, respectively, it will be appreciated that the slits could be formed in any configuration which provide a grasping area. Thus, the slits, for example, could be arced It will be appreciated that the backing layer for the collar drape 10′ can be provided with the slits 25a,b as well.

### Use of the Collar Drape

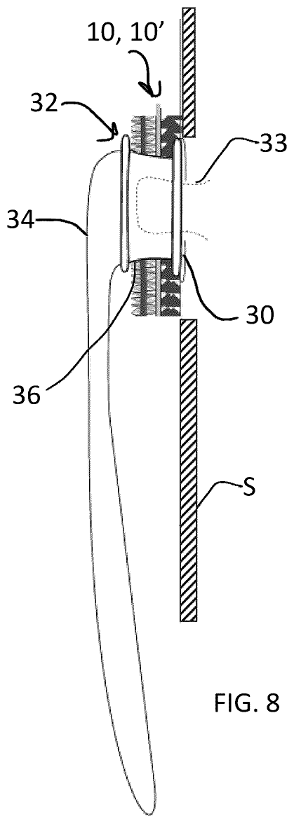

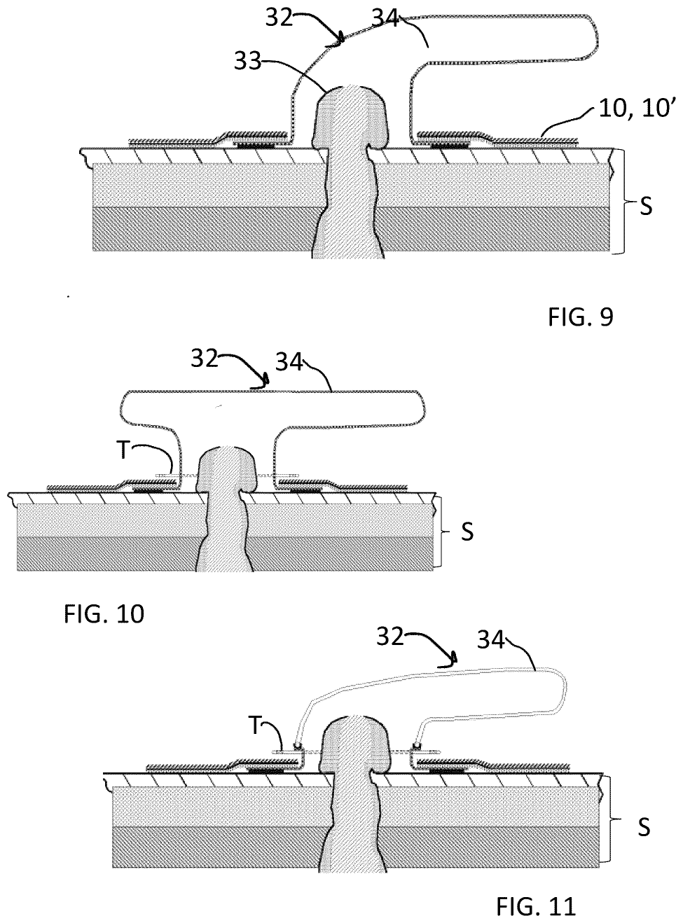

As shown in FIGS. 8-11, in use, the base 30 of the ostomy appliance 32 is adhered to the wear's skin S. In FIGS. 9-11, the skin S is shown on an enlarged scale relative to the collar drape 10 and the ostomy appliance 32 to illustrate the three layers of the skin, namely, the stratum corneum (the skin's outermost layer), the epidermis, and the dermis. The ostomy bag/pouch 34 is secured to the base 30 at a point of transition from flange to neck to bag/pouch such that the wearer's stoma 33, preferably, extends into the interior of the bag/pouch 34. As shown in FIG. 8, the ostomy appliance 32 comprises a neck 36 extending from the flange 30 with the bag/pouch 34 being connected to a free end of the neck. However, the bag/pouch 34 could be directly connected to the flange 30, which would eliminate the need for a separate neck 36 (as shown schematically in FIGS. 1A, 9, and 10). As noted above in FIG. 2, the collar drape opening 16 in the body 12 of the collar drape 10 is sized to receive the neck of the ostomy appliance (or other connection of the bag/pouch to the ostomy appliance flange).

In use, if the ostomy appliance is a two-part appliance, the ostomy appliance flange is placed on the wearer's abdomen. The collar drape 10, 10′ is then positioned over the flange, with the neck (or connector) of the ostomy appliance extending through the collar drape opening 16, 16′, and adhered to the wearer's abdomen. Thus, as apparent from in FIG. 7, the collar drape 10 will overlie the flange 30 of the ostomy appliance. With the collar drape 10 in place, the bag/pouch 34 can be secured to the neck 36 of the ostomy appliance. If the bag/pouch 32 is connected directly to the flange (i.e., in a one-piece ostomy appliance), then the bag/pouch can be pulled through the collar drape opening 16, 16′ of the collar drape 10, 10′ after the ostomy appliance has been positioned about the stoma and adhered to the wearer's abdomen. Alternatively, the bag/pouch 34 could be pulled through the collar drape opening 16, 16′ and the collar drape could be secured to the flange 34 prior to positioning the ostomy appliance about the stoma. In this manner of application, the collar drape and ostomy appliance would be applied together.

As noted above, the release liner 24 is preferably scored or perforated (as at 24a) such that a portion of the release liner 24b (FIG. 3) around the collar drape opening 16 can be removed prior to removal of the remainder of the release liner. This portion 24b of the release liner is sized to cover a portion of the flange 30 surrounding the connection neck of ostomy appliance. The removal of this border section around the collar drape opening 16 also allows for the material 22 that otherwise overlies the border to stretch to fit about the neck of the ostomy appliance and to allow for the bag/pouch to pass through collar drape opening 16.

The collar drape 10, 10′ is applied to the wearer's abdomen after the ostomy appliance flange (if the ostomy appliance is a two-part appliance) or after the ostomy appliance itself is positioned and adhered to the wearer's abdomen about the stoma 33. Initially, the portion/ring 24b of the release liner surrounding the collar drape opening 16, 16′ is removed to expose the adhesive in a border area about the collar drape opening 16, 16′. If the appliance is a two-part appliance, the collar drape is positioned to align the collar drape opening 16, 16′ with the neck of the flange, and the collar drape is then applied so that the adhesive of the collar drape in the border area around the collar drape opening 16 secures the collar drape to the flange about the neck 36. If the appliance 32 is a one-piece appliance, the bag/pouch 34 will need to be passed through the collar drape opening 16, 16′ of the collar drape 10, 10′, and then the border of exposed adhesive surrounding collar drape opening 16, 16′ can be pressed against the flange of the appliance 32 to partially adhere the collar drape to the flange 30. As noted above, the collar drape opening 16, 16′ is smaller, in at least one dimension, than the neck of the ostomy appliance. Thus, the material 22 of the collar drape 10 will stretch as the collar drape is applied to the appliance. This stretching of the collar opening will ensure a tight fit of the collar drape opening 16, 16′ about the neck of the appliance.

In a preferred embodiment, the release liner 24 is split vertically (along a line parallel to the vertical axis VA to define left and right sides of the release liner. Once the collar drape 10, 10′ is initially applied to the ostomy appliance flange 30, a first side (either the left or right side) of the release liner can be removed to expose the adherent layer 28. This first side is preferably lightly applied to the wearer's skin (to the wearer's right for the right side of the collar drape and to the left for the left side of the collar drape). Then the same procedure is carried out on the second side. Namely, the remaining release liner is removed, the second side of the collar drape is lightly applied to the wearer's skin.

Once both sides of the collar drape are initially applied to the skin, a first (e.g., right) half of the collar drape can be lifted off the skin and then stretched, e.g., to the right, and then adhered to the skin in a manner that will smooth the first (e.g., right) side of the collar drape against the skin. Then the second (e.g., left) side of the collar drape is lifted, stretched, and reapplied in the same manner. The collar drape is versatile and adapts to a multitude of applications methods.

Due to the elasticity of the collar drape 10, 10′, when the collar drape is applied in a stretched condition, as described above, the collar drape 10, 10′ will be in tension and in this state, will generate compressive forces that are applied to the ostomy appliance 32 at the neck 36. These compressive forces enhance the seal of the ostomy appliance with the wearer's skin and thus reduce the potential of leakage at the stoma/flange interface. To the extent there might be any leakage from the appliance, any leakage accumulates and is sealed underneath the device 10. This is due in part to the size of the collar drape opening 16, 16′ relative to the neck 9, 36 of the appliance. That is, the fact that the collar drape opening 16, 16, is smaller, in at least one direction, than the outer circumference of the ostomy appliance neck enhances the seal of the collar drape about the neck, and this seal is then further enhanced by the compressive forces generated when the collar drape is under compression (i.e., worn in a stretched condition). As noted above, the arms 14 can be of different lengths. If the arms 14 are of different lengths, the forces generated by the two arms when they are stretched may be different which could cause the collar drape 10 to pull to the side with the longer arm. This imbalance in forces could be compensated for by increasing the width of the shorter of the arms. However, if the arms are to be of the same width, then it is preferred that the arms 14 be of the same length. As noted above the arm 14b′ of the collar drape 10′ (FIG. 4) is shorter than the arm 14a. Accordingly, the arm 14b′ is wider (top-to-bottom) than the arm 14a′, such that the two arms define approximately equal areas. This will insure that the two arms 14a′, 14b′ will generate generally equal forces.

As seen in FIG. 6, the body 12 of the collar drape 10 is sized, and the collar drape opening 16 is positioned on the body 12, such that the collar drape body 12 will be positioned between the ostomy bag/pouch 34 and the wearer's skin for a large majority of the length of the bag/pouch 34. This can increase the comfort for the wearer, as then the bag/pouch will not rub against the wearer's skin.

Additionally, the material 22 of the collar drape can frictionally stabilize the bag/pouch 34. To facilitate this, the collar drape is formed such that the machine direction of the material 22 is generally parallel to the horizontal axis HA of the collar drape. As noted above, the brushing (used to form the upper pile 26b) is in the machine direction, and hence, is generally parallel to the horizontal axis HA of the collar drape 10. When the upper pile is formed in this fashion, the pile feels smooth when a finger is moved across the collar drape in a direction generally parallel to the vertical axis VA. However, when a finger is moved across the collar drape in a direction generally parallel to the horizontal axis HA, the upper pile 26a feels rough. Hence, if the bag/pouch 34 has a plush surface (as they often do for purposes of wearer comfort), the pile 26b of the collar drape can engage the plush surface of the bag/pouch forming a frictional contact of the bag/pouch with the collar drape to reduce movement of the bag/pouch relative to the wearer (in the machine direction). This will also help increase the wearer's comfort, especially if the wearer is wearing loose clothing which might not otherwise restrain movement of the bag/pouch.

In addition, the collar drape 10, 10′ can be provided with one or more straps 38 (FIG. 7). The straps are preferably elastic, and comprise a central portion 38a and opposed ends 38b which have a hook type surface to engage the plush surface of the collar drape 10. The strap(s) 38 thus will be removably secured at ends 38b to the collar drape by a hook-and-loop type connection. The strap 38 preferably has a length greater than the side-to-side width of the flange 30 of the ostomy appliance. Thus, the strap can be positioned between the bag/pouch 32 and the collar drape immediately under the neck, as shown in FIG. 7. In this position, the elasticity of the strap will add additional pressure to the flange 30 of the ostomy appliance to enhance the adherence of ostomy appliance to the wearer's abdomen. In this position, the strap 38 will be below the neck of the ostomy appliance and between the ostomy bag/pouch 34 and the body 12 of the collar 10. In an alternative, the strap 38 can extend over the bag/pouch 34 to help secure the bag/pouch against the wearer, and if desired, support the bag/pouch in a desired orientation on the wearer. In this instance, the strap would not be exerting any compression or forces on the ostomy bag/pouch, but rather would simply be used to prevent the bag/pouch from moving pendulously (i.e., from swinging) relative to the collar drape 10.

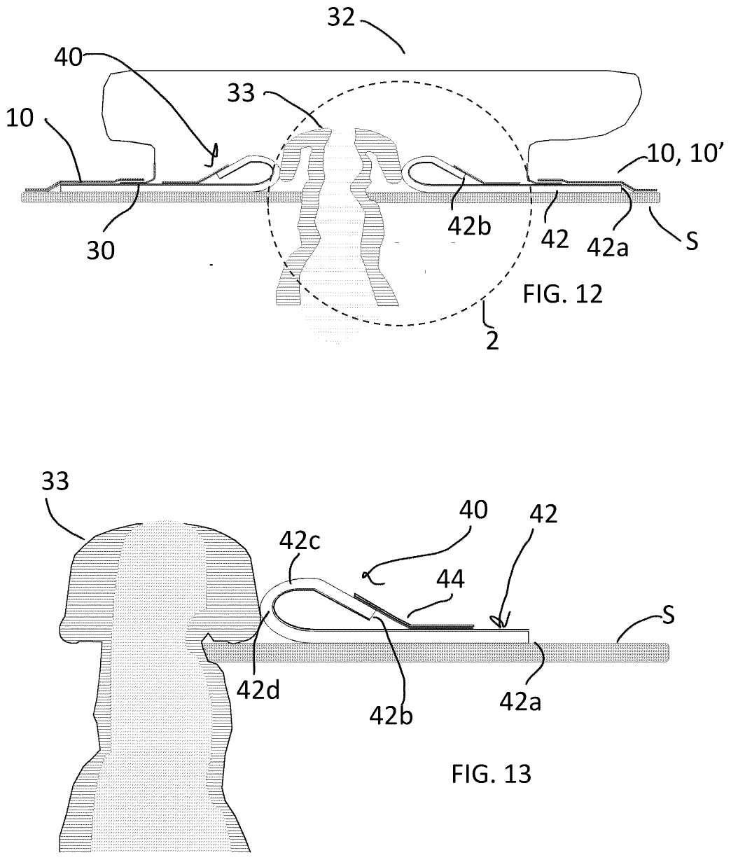

In FIGS. 12-15, an optional barrier is shown being used in conjunction with the collar drape 10, 10′. A first barrier 40 is shown in FIGS. 12 and 13. The barrier 40 comprises a barrier base 42 having a silicone gel adhesive on a bottom side to adhere the barrier base to the user's skin. This barrier base 42 can, for example, be made from a polyurethan film, such as the Medical Film 9832F available from 3M. This film is translucent and fluid resistant, is breathable, and has a high moisture transmission rate (MVTR) of about 2900 gm/sqm/day (as measured via the inverted cup or bottle method). The silicone gel adhesive can be applied in a heavy coat layer, i.e., about 250 to 1500 gm/m². The barrier base 42, which when fully flat, in plan view, can define a circle having an outer edge 42a and an inner edge 42b. The inner edge, in plan view, defines a barrier opening (which can be circular) and which has a diameter smaller than the diameter of the stoma 33. In use, as shown in FIGS. 12-13, the inner edge 42b of the barrier 40 is folded outwardly such that an inner portion 42c of the barrier base 42 overlies itself. The inner portion 42c is held in its folded or rolled-over position, for example, by means of an adhesive film 44 (i.e., a single annular adhesive film or two or more strips of adhesive film) which is adhered to the overturned inner portion at the inner end and to a main portion of the barrier base at outer end. This folded or rolled-over inner portion 42c defines a smooth inner surface 42d which forms a circle sized to engage the stoma 33 about the perimeter of the stoma, as seen in FIGS. 12-13. As can be appreciated, in view of the folding or rolling over of the inner edge 42b to form the inner portion 42c, the silicone gel adhesive that coats the bottom of the barrier base will engage the stoma 33, and will thus form a seal directly with the stoma 33 at the inner surface 42d. The folded or rolled-over inner edge of the barrier could be held in position by other means, such as by a direct adhesive securement of the inner edge to top surface of the barrier.

Returning to FIG. 12, the main portion of the barrier base extends outwardly from the inner portion a sufficient distance, such that the barrier will extend beyond the perimeter the flange 30 of the ostomy appliance 32, such that the ostomy appliance flange 30 is fully adhered to the barrier base 42 and at least a portion of the body 12 of the collar drape 10 is adhered to the barrier base 42. Thus, as can be appreciated, in use, the barrier 40 is applied to the wearer's skin, and the ostomy appliance 32 and collar drape 10 are applied over the barrier 40. The barrier 40 forms a tight (but not constricting) seal with the stoma to reduce the likelihood of output from the ostomy from contacting the wearer's skin.

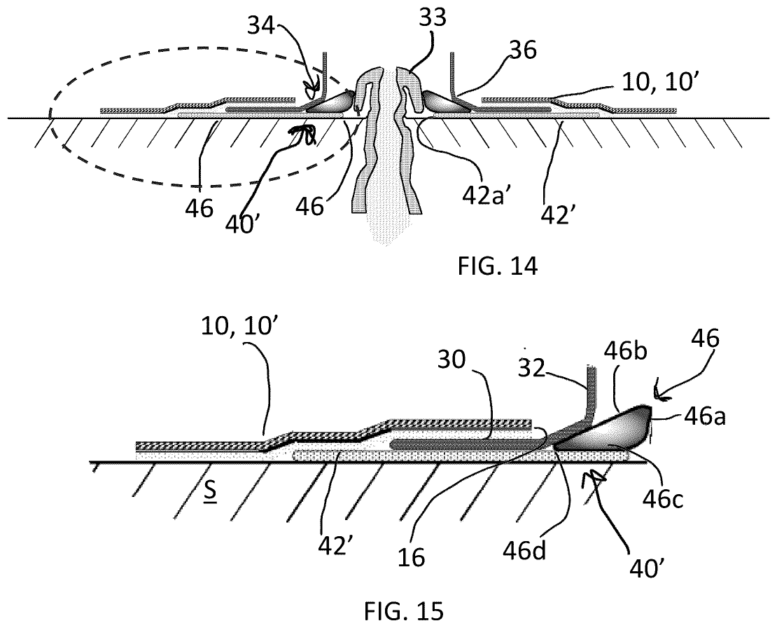

A second barrier 40′ is shown in FIGS. 14 and 15. This barrier 40′ is also designed for use with a one-piece ostomy appliance, but could be used with a 2-piece ostomy appliance. The barrier 40′ comprises an annular base 42′ having a central barrier opening 42a′ that is slightly larger than the stoma 33. Thus, unlike the base 42 of the barrier 40, the barrier base 42′ does not seal against the stoma. Rather, the second barrier 40′ also includes a ring 46 formed, preferably, from silicone, and in particular, a silicone hydrogel. The ring 46, in cross-section, is generally triangular in shape, and comprises a circumferential inner wall 46a which, in use, is generally vertical (or generally parallel to the wall of the stoma), a downwardly and outwardly sloping upper wall 46b and a bottom surface 46c. The upper and bottom walls 46b and 46c meet at a circumferential outer edge 46d (which could be an apex). The ring 46 is applied over the annular base 42′, which in turn has been applied to the skin with a silicone gel adhesive. The central barrier opening 42a′ of the barrier base, as noted, is larger than the stoma. It is also larger than the diameter defined by the inner wall 46a of the ring 46. The diameter defined by the ring inner wall 46a is sized such that it will engage the outer surface of the stoma around the perimeter of the stoma when in use to form a seal with the stoma. Thus, the ring 46 extends inwardly from (or overhangs) the inner edge 42a′ of the barrier base 42′ to then form a seal with the stoma 33 to protect the peristomal skin.

The ostomy appliance 32 is applied over the barrier 40′ such that its flange 30 overlaps a portion of the upper surface 46b of the ring and extends out over the barrier base 42′ of the barrier 40′, as best seen in FIG. 15. As seen, the barrier base 42′ extends beyond the perimeter of the base 30 of the ostomy appliance. The collar drape 10 is then applied over the base 30 of the ostomy appliance 32 and the barrier base 42′. If desired, the ring 46 could be sized such that the outer circumference defined by the outer edge 46d of the ring 46 is smaller than the circumference if the ostomy appliance neck. In this instance, the flange 30 of the ostomy appliance 32 will not rest on the ring 46, but instead, will adhere to the barrier base 42′. Thus, as with the barrier 40, when the barrier 40′ is used, the flange 30 of the ostomy appliance is not in direct contact with the wearer's skin. Just as when the collar drape 10, 10′ is used without the barrier 40, 40′, any leakage from the appliance will be accumulated, sealed, and contained by the collar drape 10, 10′ in a manner to avoid contact of the leakage with the wearer's skin.

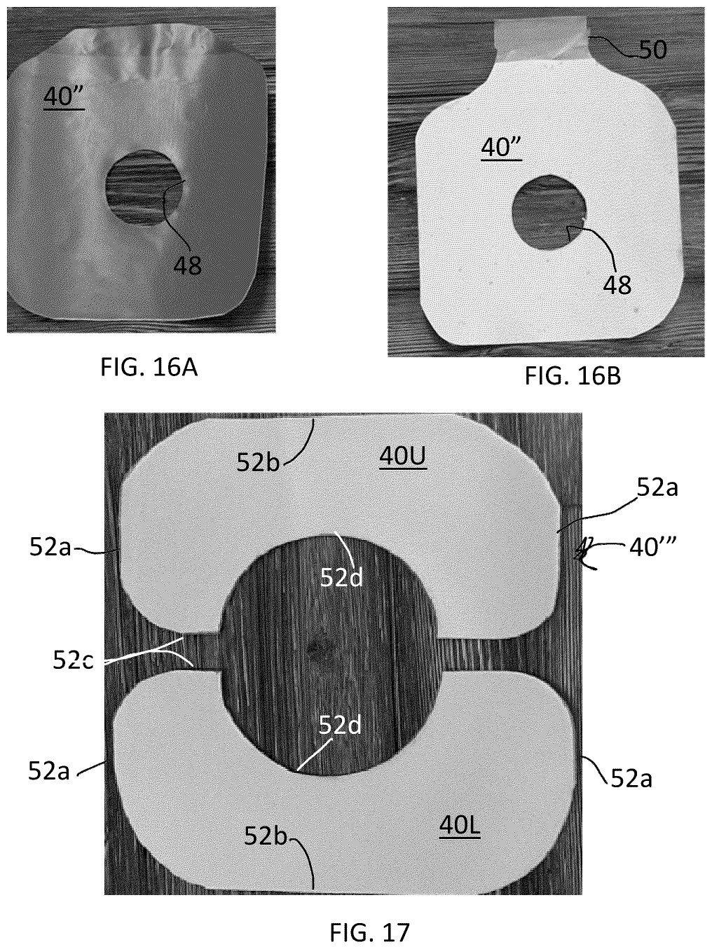

FIGS. 16A,B show two configurations of a one-piece planar barrier 40″. The two barriers are each generally square (although they could be rectangular, circular, or ovoid) and are sized to define a perimeter greater than the perimeter of the flange 30 of the ostomy appliance 32 to form an extension beyond the flange of the ostomy appliance. Preferably, the barrier 40″ is about 40% larger in area than the flange 30 of the ostomy appliance 32. The barrier 40″ is provided with a central barrier opening 48 sized to fit around a stoma. Inasmuch as stomas vary in size, the material from which the barrier 42″ is made can be cut to increase the size of the barrier opening to fit the stoma about which the barrier is to be applied. As noted, the two barriers in FIGS. 16A-B are generally identical. The main distinguishing feature is that the barrier of FIG. 16B is provided with a neck 50 which can facilitate removal of the backing layer from the material of the barrier.

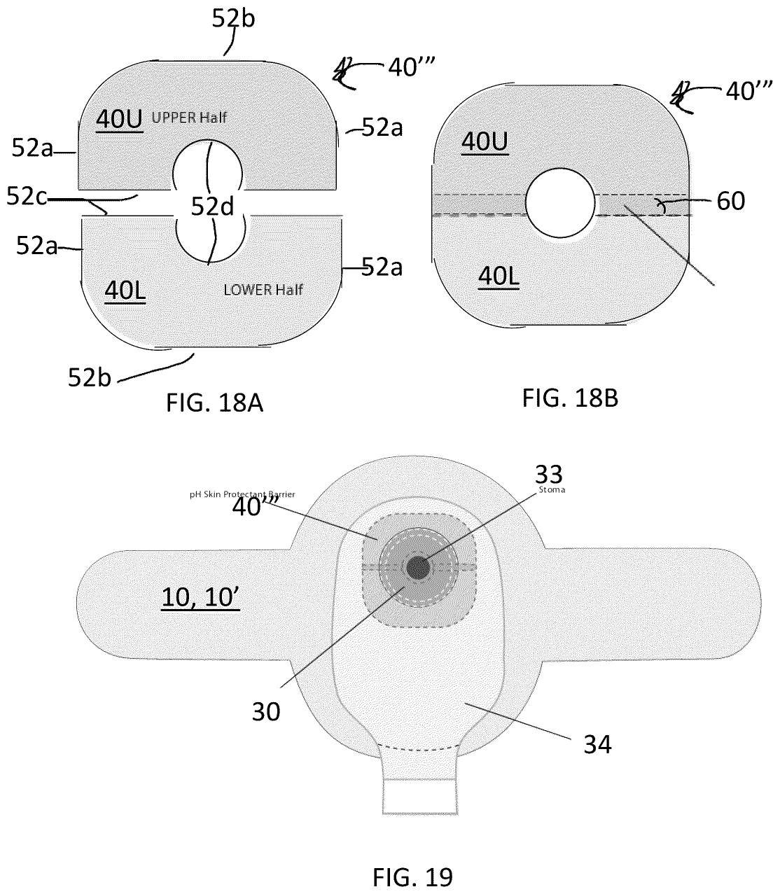

FIGS. 17 and 18A,B show two-piece barriers 40′″. The barriers 40′″ comprise an upper piece 40U and a lower piece 40L. The upper and lower pieces 40U, L can be mirror images of each other or even identical to each other. Each piece is generally rectangular in shape and comprises side edges 52a, an outer edge 52b and an inner edge 52c. An arcuate concave cutout 52d is formed in inner edges 52c of each of the upper and lower pieces 40U, L. The outer corners (i.e., between the outer edges 52b and the side edges 52a) are curved. The difference between the barrier of FIG. 17 and the barrier of FIGS. 18A,B is that in the barrier of FIG. 17, the corner at the junction of the side edges 52a and the inner edge 52c is curved, whereas in the barrier of FIGS. 17A,B, the corresponding corner is approximately a 90° corner. When the two-piece barrier 40′″ is used, the two-pieces are applied to the skin around the stoma on opposite sides of the stoma with the deepest part of the arced cutouts 52d of the two portions 40U, L adjacent the stoma. This will, most likely, result in an area 60 of overlap between the two portions, as seen in FIG. 18B. Depending on the amount of overlap, the perimeter of the two cutouts surrounding the stoma could, in combination, define a circle. For a smaller stoma, the two cutouts, in combination would form a cat's eye shape or connected parentheses (i.e., “( )”). As the overlap increases, and the shape defined by the perimeter of the two cutouts, in combination, changes from a circle to a cat's eye shape, the amount of exposed skin adjacent the stoma will be increased. Thus, the two portions of the barrier 40′″ are preferably applied above and below the stoma.

Like the barrier 40′, the barriers 40″ and 40′″ are intended to be used with a ring, such as the ring 46, made from a silicone hydrogel that will cover the exposed skin between the barrier and the stoma and which will form a seal with the stoma. This ring can be supplied in ring form, or can be provided as a pliable material that is formed into a ring around the stoma, and will thus form a seal from the stoma to the barrier, with a portion of the ring covering an inner edge of the barrier.

Each of the barriers 40, 40′, 40″, and 40′″ is made from a polyurethane (PU) substrate of that is preferably about 100 microns thick. A bottom surface of the substrate is coated with a silicone gel or acrylate gel of approximately 250 grams per square meter and which has an adhesion of 1.6 N per 25 mm. At least the bottom surface (i.e., the gel adhesive) of the barrier is covered with a release liner. This bottom release liner can be made from an LDPE. The top surface of the barrier can also be covered with a release liner to provide additional protection to the top surface of the barrier. If the top surface is also covered with a release liner, this upper release liner can be, for example, a 63# kraft siliconized release liner.

Although the barriers are shown used in conjunction with a collar drape 10, 10′, the barriers could be used without a collar drape in which case, the ostomy appliance 30 is applied directly to the barrier 40, 40′, 40″, 40′″. Thus, a wearer can use the barrier and collar drape in three ways: (1) the collar drape 10, 10′ by itself, (2) the barrier 40, 40′, 40″, 40′″ by itself, or (3) the barrier and collar drape can be used together (or in combination). In the first instance, the collar drape is applied directly to the patient, and the ostomy appliance is applied (adhered) to the collar drape; in the second instance, the barrier 40, 40′, 40″, 40′″ is applied directly to the patient, and the ostomy device is applied (adhered) to the barrier; and in the third instance, the barrier 40, 40′, 40″, 40′″ is applied to the patient, the ostomy appliance 30 is applied to the patient over the barrier, and the collar drape 10, 10′ is applied over the flange 30 of the ostomy appliance. The manner of use will be determined by the wearer's needs. Thus, an ostomy appliance can be packaged with a collar drape and a barrier, such that the three items are packaged or sold as a kit, giving the patient the option to use the collar drape and barrier together or to use just one of them.

FIG. 19 shows a complete assembly of a collar drape 10, 10′ and barrier (specifically the barrier 40′″) with an ostomy appliance in place. Initially, the barrier is applied to the skin around the stoma 33. The ring 46 of the barrier 40′, 40″ is then formed or positioned around the stoma to complete the seal surrounding the stoma. The flange 30 of the ostomy appliance is cut such that its center opening is sized to seal against the ring 46, and as sized, is adhered to the barrier 40, 40′, 40″, 40′″. As seen in FIG. 19, the barrier extends beyond the perimeter of the ostomy appliance flange 30. The collar drape 10 is then applied over the ostomy appliance flange as described above. As can be appreciated, the collar drape 10 will be adhered to the ostomy appliance flange, the barrier, and beyond the perimeter of the barrier, to the wearer's skin. As can be appreciated, the flange of the ostomy appliance is adhered to the barrier-the flange of the ostomy appliance does not contact the wearer's skin.

As can be seen in FIGS. 11-18, when the barrier 40, 40′, 40″, 40′″ is used, the collar drape 10, 10′ does not seal (or does not need to seal) against ostomy appliance in the same manner as when a barrier is not used. That is, the collar drape opening 16, 16′ of the collar drape 10, 10′ can be larger than the neck of the ostomy appliance. Instead, the barrier 40, 40′, 40″, 40′″ forms a seal with the stoma (via the barrier itself, as with barrier 40) or using a ring (such as the silicone ring 46), hence the collar drape need not tightly fit about the neck of the ostomy appliance.

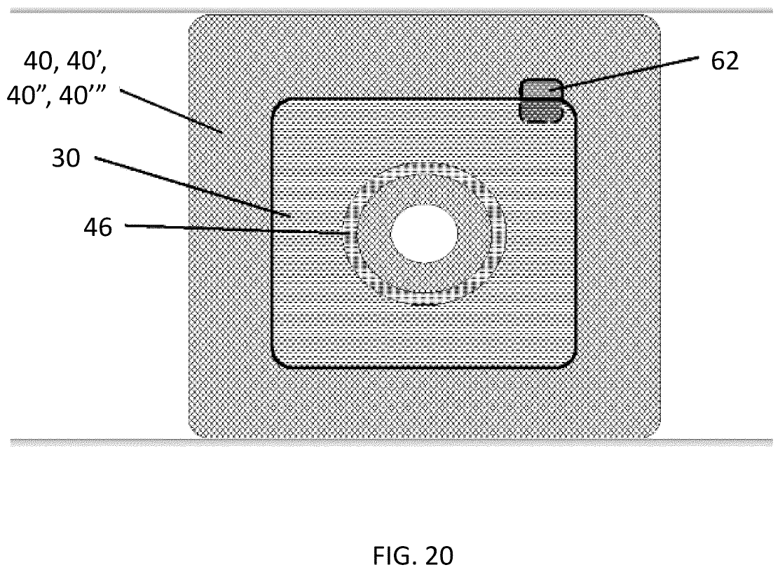

The barrier is intended to be worn for an extended period of time, i.e., at least several days, and up to several weeks, or even, for a month or more. The ostomy appliance will need to be changed before the barrier is changed. The adhesive of the barrier (holding the barrier to the wearer's skin) is stronger than the adhesive used on the ostomy flange (which is typically a weak adhesive). Thus, the ostomy appliance can be removed from the barrier (after the collar drape has been removed) without affecting the adhesion of the barrier to the wearer's skin. FIG. 20 shows a lift tab 62 which is applied to the flange 30 of the ostomy appliance 32. The lift tab 62 extends beyond the flange 30, and can be used to facilitate removal of the ostomy appliance from the barrier. The lift tab 62 is applied to the ostomy appliance flange 30 by means of an adhesive that is stronger than the adhesive which adheres the flange 30 to the barrier, such that the lift tab can effectively be used to at least initiate separation of the flange 30 from the barrier.

Finally, the barrier can be washed, allowing for the wearer to bath or shower while the barrier is still being worn. When the barrier needs to be changed, the adhesive allows for the barrier to be removed without damage to the skin.

As can be appreciated, the barrier provides improved protection for the peristomal skin because the ostomy appliance can seal more securely against the barrier than it can against the wearer's skin. This is due to the fact that the barrier will present a more uniform and planar surface to which the ostomy appliance flange can adhere. This will present a more comfortable situation for the wearer. Further, the barrier material is fluid impervious and can withstand the pH of the ostomy fluid. Thus, if there is any leakage from the ostomy appliance, the leakage will seep onto the barrier which will protect the wearer's skin, and will substantially prevent the ostomy fluid from contacting the wearer's skin. Although the barrier is shown, in FIG. 19, to be used with a collar drape, the barrier could be used by itself (i.e., without the collar drape). Patients who are relatively thin, or who present a generally flat peristomal surface (even when sitting or standing) may not need the collar drape, and thus may prefer to use the barrier without the collar drape.

Importantly, the collar drape 10, 10′ does not interfere with the application, use, or servicing the ostomy appliance 30 or with any additions the appliance manufacturer might include with the ostomy appliance. For example. Many ostomy appliances include tabs T (FIGS. 10 and 11) to which a belt can be secured. The collar drape 10, 10′, as seen in FIG. 10 will lie between the tab T and the appliance flange.

The collar drape or barrier can be provided with various enhancements which can be included individually or in combination. For example:
The collar drape or barrier can be impregnated with a pH sensitive compound which will cause the collar drape or barrier to change color in the presence of fluids from the stoma. In this matter, the collar drape or barrier would then include leak detection capabilities.
The collar drape can be provided with indicia to determine how far the collar drape 10 is being stretched during application. For example, it could be provided with parallel lines which, as the collar drape is stretched, are pulled apart. When the lines are a determined distance (such as a finger width) apart, the wearer knows that the device has been stretched an appropriate amount.

We have found that the collar drape 10. 10′ provides increased satisfaction and comfort for those who have ostomies. Further, the material 22 from which the collar drape 10 is made is washable. Thus, as with the barrier, the wearer need not remove the collar drape 10 to bath or shower. The collar drape 10, 10′ can thus be worn for at least several days, and need only be changed when the wearer changes the ostomy appliance. If desired, the wearer could wash and reapply the collar drape 10, to reuse the same collar drape with a new ostomy appliance.

As various changes could be made in the above constructions without departing from the scope of the invention, it is intended that all matter contained in the above description or shown in the accompanying drawings shall be interpreted as illustrative and not in a limiting sense. For example, although the collar drape 10 is preferably made from the material 20, it could be made from any adhesive material which can adhere to the wearer's skin without damaging the skin. Although shown with arms, the collar drape could be provided without arms 14. These examples are merely illustrative.

1. A collar drape for use with an ostomy appliance to enhance a seal of an ostomy appliance about a stoma of an ostomy, the ostomy appliance comprising a flange adapted to adherable to skin and a bag/pouch operatively connected to the flange at a neck; the collar drape:
a body made from an elastic stretchable flexible material; the flexible material comprising a top layer, a reinforcing layer, and an adherent layer; the adherent layer comprising an adherent adapted to secure the drape to skin of the wearer; and
the collar drape further including a collar drape opening in the body; the collar drape opening being sized to be equal to or slightly smaller than an outer diameter of the neck of the ostomy appliance; the collar drape opening being generally centered vertically along a horizontal axis (HA) or being vertically spaced above the horizontal axis (HA) of the body in which instance a distance (D1) from a center of the collar drape opening to a bottom edge of the collar drape is greater than a distance (D2) from the center of the collar drape opening to a top edge of the collar drape.

2. The collar drape of claim 1 wherein the collar drape opening is sized to permit the ostomy bag/pouch to pass through the collar drape opening during application and use of the collar drape, and wherein the neck of the ostomy appliance must extend through the collar drape opening during application of the collar drape.

3. The collar drape of claim 1 wherein an inner edge of the collar drape opening is adapted to surround the neck of the ostomy appliance with which the collar drape is used to form a seal between the ostomy appliance and the collar drape about the neck of the ostomy appliance.

4. The collar drape of claim 1 comprising a release liner removably adhered to the adherent layer; the release liner defining a removable border in the release layer surrounding the collar drape opening; whereby removal of the border enables the collar drape opening to expand when the drape is positioned about an ostomy appliance while the release liner remains in place over the remainder of the collar drape.

5. The collar drape of claim 1, further including arms which extend from opposite sides of the drape body.

6. The collar drape of claim 1 wherein the lower distance (D1) from the center of the opening to the bottom edge of the drape is equal to at least 70% of a length of the ostomy bag/pouch from a stoma receiving aperture of the ostomy bag/pouch to a bottom of the ostomy bag/pouch.

7. The collar drape of claim 1 wherein the drape has a plush/brushed upper surface.

8. The collar drape of claim 7 further including a strap which is removably securable to the collar drape; the strap comprising an elastic central section and opposed ends which are adapted to removably engage the plush surface of the collar drape; whereby the strap can be applied to the collar drape either (1) to extend between the ostomy bag/pouch and the collar drape to vertically support to the ostomy bag/pouch in use or (2) to extend over the ostomy bag/pouch to restrain movement of the ostomy bag/pouch relative to the collar drape.

9. The collar drape of claim 8 wherein the strap is elastic and has a length at least when stretched that is greater than a side-to-side width of a base of the ostomy appliance with which the collar drape is used, wherein when the strap is applied to the collar drape below the neck of the ostomy appliance, the strap will add pressure to the base of the ostomy appliance below the neck.

10. The collar drape of claim 1 wherein due to its elasticity, when applied to a wearer in a stretched state, the collar drape will generate compressive forces which are applied/directed to the flange and neck of the ostomy appliance to enhance a seal of the ostomy appliance about the stoma.

11. A barrier for use in conjunction with an ostomy appliance, the ostomy appliance comprising a flange having an adhesive coated bottom surface and an ostomy bag/pouch, the barrier comprising:
a barrier base having an upper surface, a lower surface, and a barrier opening spaced from a perimeter of said barrier; said barrier base being formed from a material to which said ostomy appliance flange can be removably adhered; said barrier base defining a perimeter greater than a perimeter of said ostomy appliance flange, such that when said ostomy appliance flange is adhered to said barrier base, said barrier base will extend beyond the perimeter of said ostomy appliance flange; and
a barrier adhesive covering the bottom surface of said material; said barrier adhesive being stronger than said adhesive of said ostomy appliance flange, whereby the ostomy appliance can be removed from the barrier without removing the barrier from the wearer.

12. The barrier of claim 11 wherein the barrier opening is defined by a rolled or folded over inner portion of the barrier defining an inner surface which is sized and adapted to engage the stoma about the periphery of the stoma when applied, whereby, in use, the adhesive of the barrier is adapted to seal against the stoma.

13. The barrier of claim 11 wherein the barrier comprises an annular ring having an inner surface defining an opening sized, shaped, and adapted to engage and seal against a stoma, and a bottom surface having an outer diameter greater than a diameter of said barrier opening and an inner diameter no greater than the diameter of said barrier opening, said ring being adapted to be adhered to said upper surface of said barrier.

14. The barrier of claim 11, wherein the barrier adhesive is a silicone gel or acrylate gel.

15. The barrier of claim 11 wherein said barrier further comprises a lower release liner sized to cover and protect said barrier adhesive, wherein said lower release liner preferably formed from siliconized paper.

16. The barrier of claim 11 wherein said barrier further comprises an upper release liner covering an upper surface of said barrier; wherein said upper release liner is preferably formed from a low-density polyethylene (LDPE).

17. The barrier of any claim 11 wherein said barrier comprises a first portion and a second portion; the first and second portions each having an inner edge defining an arcuate cutout; the cutouts of said first and second portions, in combination, defining the opening of said barrier.

18. The collar drape of claim 11 wherein the barrier is split in two at least partially, such that the barrier opening is formed in two halves.

19. The barrier of claim 18 wherein the split extends from one edge to another of the barrier, such that the barrier is formed of two pieces.

20. A kit for use with an ostomy appliance, the ostomy appliance comprising an ostomy appliance flange and a bag/pouch, the kit comprising a collar drape and a barrier;
the collar drape comprising:
a collar drape body formed from an adhesive assembly, the adhesive assembly comprising an elastic stretchable flexible material; the flexible material comprising a top layer, a reinforcing layer, and an adherent layer; the adherent layer comprising an adherent adapted to secure the drape to skin of the wearer and/or to the barrier; and
the collar drape body defining an collar drape opening sized to be equal to or slightly smaller than an outer diameter of the neck of the ostomy appliance with which the collar drape is to be used; the collar drape opening being generally centered vertically along the horizontal axis (HA) or being vertically spaced above a horizontal axis (HA) of the body in which instance a distance (D1) from a center of the collar drape opening to a bottom edge of the collar drape is greater than a distance (D2) from the center of the collar drape opening to a top edge of the collar drape; and
the barrier comprising:
a barrier base having an upper surface, a lower surface, and a barrier opening spaced from a perimeter of said barrier; said barrier base being formed from a material to which said ostomy appliance flange can be removable adhered; said substrate preferably being formed from a film comprised of polyurethane, polyethylene, or a combination thereof; said substrate defining a perimeter greater than a perimeter of a flange of the ostomy appliance with which the barrier is to be used, such that when said ostomy appliance base flange is adhered to said substrate, said substrate will extend beyond the perimeter of said base flange; and
a barrier adhesive covering the bottom surface of said substrate; said barrier adhesive being stronger than said adhesive of said ostomy appliance flange, whereby the ostomy appliance can be removed from the barrier without removing the barrier from the wearer.

21. The kit of claim 20 wherein only the barrier is used with ostomy appliance, wherein only the collar drape is used with the ostomy appliance, or wherein both said barrier and collar drape are used with said ostomy appliance.

22. The barrier of claim 21 wherein when the barrier is used in conjunction with the collar drape, the collar drape opening is larger than the barrier opening but smaller than an outer diameter of the barrier.
